(19) United States
(12) Patent Application Publication
(10) Pub. No.: US 2007/0026060 A1
(43) Pub. Date: Feb. 1, 2007

(54) Method for treating advanced ovarian cancer

(76) Inventors: Ronald D. Alvarez, Birmingham, AL (US); J. Michael Straughn JR., Birmingham, AL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/408,201

(22) Filed: Apr. 20, 2006

Related U.S. Application Data
(60) Provisional application No. 60/674,188, filed on Apr. 21, 2005.

(51) Int. Cl. A61K 9/127; A61K 31/704

(52) U.S. Cl. 424/450; 514/34

(57) ABSTRACT

A method for treating advanced epithelial ovarian cancer patients who attain a clinically-defined complete response after initial platinum/paclitaxel-based chemotherapy is described. The method involves administering a chemotherapeutic agent, such as doxorubicin, entrapped in liposomes.

### CROSS-REFERENCE TO RELATED APPLICATIONS

This application claims the benefit of U.S. Provisional Application No. 60/674,188, filed Apr. 21, 2005, incorporated herein by reference in its entirety.

### TECHNICAL FIELD

The subject matter described herein relates to a method of treating advanced ovarian cancer in subjects previously treated with platinum/paclitaxel-based chemotherapy. More specifically, the subject matter relates to a method of treating advanced ovarian cancer in treatment-experienced patients with liposome-entrapped doxorubicin.

### BACKGROUND

Ovarian carcinoma is the leading cause of death in patients with gynecologic malignancies. The estimated incidence and mortality for 2005 are 22,220 and 16,210 respectively (Jemal A., et al., CA Cancer J. Clin., 55(1):10-30 (2005)). The majority of women diagnosed with advanced epithelial ovarian cancer will demonstrate a clinically-defined response to platinum/paclitaxel chemotherapy (Cannistra, S. A., N. Engl. J. Med., 351(24):2519-29 (2004)). The overall response rates can exceed 70% with complete clinical response rates of approximately 30-50% for patients with suboptimally resected disease (McGuire, W. P. et al., N. Engl. J. Med. 334(1):1-6 (1996); Muggia, F. M., et al., J. Clin. Oncol., 18(1):106-15 (2000)). Unfortunately, these responses are not durable with a majority of patients demonstrating recurrence of disease (McGuire, W. P. supra; Muggia, F. M., supra; Ozols, R. F., et al., J. Clin. Oncol., 21(17):3194-200 (2003)).

Several strategies have been explored to improve outcomes and prognosis of patients with advanced epithelial ovarian cancer. However, randomized trials have failed to demonstrate an overall survival benefit with prolonged chemotherapy in these patients, and additional cycles of chemotherapy have been associated with significant toxicity (Hakes, T. B., et al., Gynecol. Oncol., 45(3): 284-9 (1992); Bertelsen, K. et al., Gynecol. Oncol., 49(1): 30-36 91993); Lambert et al., Int. J. Gynecol. Cancer, 5(1): 41 (1995)). It is desirable to provide treatment regimens for patients with a complete clinical response to standard chemotherapy to consolidate the initial response and to prolong the disease-free interval.

The anthracycline antibiotic doxorubicin possesses a broad spectrum of antineoplastic action that has been used in multiple solid tumors such as breast, ovary, bladder and thyroid. The conventional formulation of doxorubicin is rapidly cleared from the bloodstream and has a large distribution volume. A liposome-entrapped doxorubicin, Doxil®, provides an increased blood circulation time of the drug, reduces the nonspecific delivery to normal tissues. and avoids high plasma levels responsible for toxicity. These pharmacologic activities improve the specificity for tumors by allowing higher drug levels to eventually extravasate through the abnormally permeable vessels characteristic of many tumors. The liposome entrapped doxorubicin Doxil® has demonstrated activity in epithelial ovarian cancer as a first or second line agent (Gordon, A. N. et al., Gynecol. Oncol., 95(1):1-8 (2004); Rose, P. G. et al., Abstract No. 1531, ASCO 2000; Gibbs et al. Abstract No. 1539, ASCO 2000).

There remains a need for a consolidation strategy following the attainment of a clinically defined complete response to initial platinum/paclitaxel-based chemotherapy in patients with advanced ovarian cancer.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a method of treating advanced ovarian cancer in a patient previously treated with platinum/paclitaxel-based chemotherapy and having a defined complete response to such treatment is provided. The method comprises administering an anthracycline entrapped in liposomes, optionally having an outer surface coating of hydrophilic polymer chains.

In another aspect, a consolidation treatment strategy for a patient diagnosed with advanced ovarian cancer is provided. The strategy comprises treating the patient with platinum/paclitaxel-based chemotherapy to achieve a complete response and subsequently treating the patient with an anthracycline entrapped in liposomes, optionally having an outer surface coating of hydrophilic polymer chains.

In addition to the exemplary aspects and embodiments described above, further aspects and embodiments will become apparent by reference to the drawings and by study of the following descriptions.

### BRIEF DESCRIPTION OF THE DRAWINGS

FIG. 1 shows the overall survival, in months, for patients treated with liposome-entrapped doxorubicin as detailed in Example 1.

### DETAILED DESCRIPTION

As noted above, the majority of patients with advanced epithelial ovarian cancer, despite an initial complete response to cytoreductive surgery and/or platinum/paclitaxel-based chemotherapy, experience a recurrence of disease. The present treatment method is based on the discovery that patients with suboptimally resected disease and a complete clinical response to initial chemotherapy can be treated with an anthracycline entrapped in a liposome to extend overall survival time.

Ovarian cancer can be staged according to the AJCC/TNM System that describes the extent of the primary tumor (T), the absence or presence of metastasis to nearby lymph nodes (N), and the absence or presence of distant metastasis (M). This closely resembles the system that is actually used by most gynecologic oncologists, called the FIGO system. “Advanced epithelial ovarian cancer”, as used herein, intends patients with stage III or stage IV ovarian cancer. More particularly, and in one embodiment, the term intends patients with stage IIIc or stage IV ovarian cancer, determined according to a recognized staging technique such as the AJCC/TMN or FIGO system. In patients diagnosed with stage III ovarian cancer the cancer involves one or both ovaries, and one or both of the following are present: (1) cancer has spread beyond the pelvis to the lining of the abdomen; (2) cancer has spread to lymph nodes. In stage IIIC patients, the cancer is in one or both ovaries, and one or both of the following are present: (1) cancer has spread to lymph nodes, and (2) deposits of cancer larger than 2 cm across are present in the abdomen. Patients diagnosed with stage IV have cancer in one or both ovaries. Distant metastasis (spread of the cancer to the inside of the liver, the lungs, or other organs located outside of the peritoneal cavity) has occurred. A finding of ovarian cancer cells in pleural fluid (from the cavity that surrounds the lungs) is also evidence of stage IV disease.

Patients diagnosed with advanced ovarian cancer are treated initially with surgery and chemotherapy. Debulking surgery is done to reduce the number of cancer cells that need to be destroyed by chemotherapy and therefore decrease the likelihood of the cancer developing a resistance to chemotherapy. As used herein, a patient that has been “optimally debulked” is defined as a patient having a residual tumor with a diameter of 2 cm or less, more preferably of 1 cm or less. “Suboptimally debulked” intends a residual tumor, i.e, tumor after surgical debulking, with a diameter of greater than about 2 cm, more preferably of greater than about 1 cm.

After surgical debulking, optimally or suboptimally, the current treatment protocol for stage III and stage IV ovarian cancer patients includes chemotherapy that includes a platinum chemotherapeutic agent, such as cisplatin or carboplatin. It will be appreciated that the platinum agent may be used in combination with one or more chemotherapeutic agents, such as a taxane like paclitaxel. In one embodiment, patients that have received six courses of platinum-based chemotherapy are selected for treatment.

Advanced ovarian patients treated with surgical debulking and chemotherapy comprised of a platinum-based agent may achieve a complete response. A clinically defined complete response intends (i) no evidence of cancer on physical exam within fourteen days after completion of initial chemotherapy, (ii) no evidence of residual tumor on a CT scan of the abdomen/pelvis within 60 days of completion of initial chemotherapy, and (iii) a cancer antigen-125 (CA-125) level that is less than or equal to 35 units/mL, measured in the blood or fluid from the abdominal or chest cavities.

In a study conducted in support of the method described herein, patients with advanced ovarian cancer previously treated to a clinically defined complete response with surgical debulking and a platimum-based chemotherapy were selected for treatment. As detailed in Example 1, the patients were treated with doxorubicin entrapped in liposomes having a surface coating of hydrophilic polymer chains, as exemplified by polyethyleneglycol (PEG), also referred to as pegylated liposomes. The majority of patients completed four cycles of treatment with liposome-entrapped doxorubicin given once every 28 days, with all patients receiving three of the scheduled four cycles. Table 2 below indicates that no significant toxicity (grade 4) was observed.

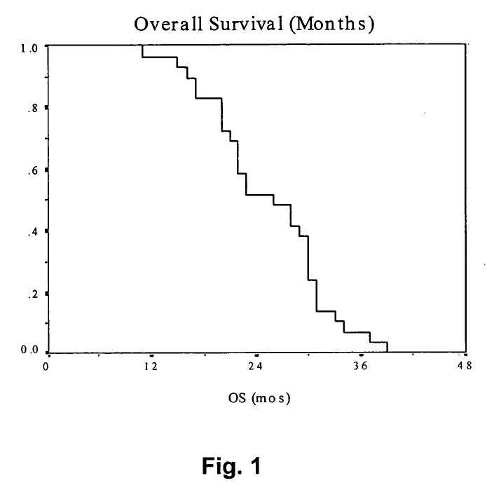

FIG. 1 shows the overall survival after treatment with liposome-entrapped doxorubicin as a consolidation agent. The patients had a progression-free interval (PFI) of about 15 months. The results show that therapy using liposome-entrapped doxorubicin is well-tolerated as a consolidation agent with an acceptable toxicity profile and provided a continued response in patients with advanced ovarian cancer.

It will be appreciated that doxorubicin is an exemplary anthracycline compound, and that other anthracyclines are contemplated, such as daunorubicin, epirubicin, and idarubicin. Similarly, the composition of the liposomal platform can be widely varied, including but not limited to, the presence or absence of an external surface coating of hydrophilic polymer chains, the polymer forming the hydrophilic polymer chains when present, the lipids forming the liposomal bilayer. The various components to form liposomes and techniques for preparation are well known in the art.

It will also be appreciated that the dose, dosing regimen, and route of administration of the liposome-entrapped anthracycline can be varied to optimize the response. The dose can be increased or decreased from that exemplified as deemed suitable for a medical provider. The interval between doses and the number of doses or ‘cycles’ of treatment can also be varied. Intravenous injection is a preferred route of administration, but any parenteral mode of delivery is contemplated.

### EXAMPLES

The following example further illustrates the aspects and embodiments described herein and is in no way intended to be limiting.

### Example 1

### Treatment of Advance Ovarian Cancer Patients

Twenty-nine patients who had histologically confirmed FIGO Stage IIIC or IV suboptimally-debulked epithelial ovarian cancer and who had achieved a clinically defined complete response to six cycles of platinum/paclitaxel-based chemotherapy were selected. Clinically defined complete response was defined as no evidence of disease on both physical exam and computerized tomography, and a CA-125≦35 units/mL. All patients had a performance status of 0-2 by Zubrod criteria. The median age was 57 (range 46-79). At the time of primary cytoreductive surgery, 26 patients had FIGO stage IIIC disease and three patients had stage IV disease. All patients had suboptimally-debulked disease. Eighteen patients had papillary serous histology, six had endometrioid, and five had mixed histology. The patient demographics are summarized in Table 1.

TABLE 1

| | n (%) |
|---|---|
| Age, Years | |
| Mean | 57.0 |
| Range | 46-79 |
| Race | |
| White | 29 (100%) |
| Black | 0 (0%) |
| FIGO Stage | |
| IIIC | 26 (90%) |
| IV | 3 (10%) |
| Histology | |
| Papillary Serous | 18 (62%) |
| Endometrioid | 6 (21%) |
| Mixed | 5 (17%) |

Patients received doxorubicin entrapped in PEGylated liposomes (Doxil®) at a one-hour infusion dose of 40 mg/m² every 28 days for four cycles. Dose reductions of 25% and treatment delays of 1-2 weeks were allowed for drug-related toxicities. Patients were followed every three months for the first two years, and then every six months until documentation of disease. Patients were removed from treatment for unacceptable toxicities. Progression-free interval (PFI) was calculated as the date of completion of primary platinum/paclitaxel-based chemotherapy to the date of recurrence. Survival analysis was calculated using the Kaplan-Meier method.

Twenty-three (23) of the 29 evaluable patients (79%) completed all four cycles of consolidation therapy with Doxil®. Due to drug-related toxicities, 14 patients required a 25% dose reduction. Dose reduction was performed for 11 patients with grade ⅔ palmar-plantar erythrodyesthesia (PPE), two patients with grade 3 myelosuppression, and one patient with grade 2 mucositis. A treatment delay was necessary for eight patients (five for PPE, two for myelosuppression, one for viral syndrome). Of note, 17 of 22 patients who required a dose reduction or treatment delay completed the scheduled four cycles of consolidation therapy. Although six patients discontinued treatment after three cycles, all patients received at least three cycles of therapy. Five patients had the last cycle discontinued for PPE, and one patient had treatment discontinued for recurrent disease.

The majority of patients (83%) in the study experienced mild toxicity with consolidation therapy with a total of 54 events noted. The events are summarized in Table 2. All toxicities were grades 1-3 with no grade 4 toxicities noted. There were no treatment-related deaths. The most common toxicities were PPE (n=16), neuropathy (n=9), nausea/vomiting (n=9), stomatitis/mucositis (n=8), and neutropenia (n=4). Of the 54 toxicities noted, only 8 (15%) were grade 3: PPE (n=4), myelosuppression (n=3), and nausea/vomiting (n=1).

TABLE 2

| | No. | (%) | 1 | 2 | 3 | 4 |
|---|---|---|---|---|---|---|
| Hematologic | | | | | | |
| Netropenia | 4 | 7.4% | 1 | 0 | 3 | 0 |
| Anemia | 0 | n/a | 0 | 0 | 0 | 0 |
| Thrombocytopenia | 0 | n/a | 0 | 0 | 0 | 0 |
| Leucopenia | 0 | n/a | 0 | 0 | 0 | 0 |
| Gastrointestinal | | | | | | |
| Stomatitis/Mucositis | 8 | 14.8% | 2 | 6 | 0 | 0 |
| Nausea/Vomiting | 9 | 16.7% | 5 | 3 | 1 | 0 |
| Constipation | 6 | 11.1% | 4 | 2 | 0 | 0 |
| Neurologic | | | | | | |
| Neuropathy | 9 | 16.7% | 6 | 3 | 0 | 0 |
| Skin | | | | | | |
| PPE | 16 | 29.6% | 4 | 8 | 4 | 0 |
| Pruritis | 2 | 4% | 1 | 1 | 0 | 0 |
| Total | 54 | 100% | 23 | 23 | 8 | 0 |

Of the 29 evaluable patients, 5 patients remain clinically without evidence of disease with a median follow-up of 35 months from the time of completion of primary chemotherapy (range, 16-42 months). Nearly 80% of patients (23 of 29) have recurred with a median time to recurrence of 12 months (range, 3-30 months). Survival analysis demonstrated a median PFI of 15 months and 2-year overall survival (OS) of 62% (18/29). The overall survival is shown in FIG. 1.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A method of treating a patient with ovarian cancer suboptimally-debulked and previously treated with a platinum chemotherapeutic agent, comprising:
administering an anthracycline compound entrapped in liposomes.

2. The method of claim 1, wherein said administering comprises administering doxorubicin entrapped in liposomes.

3. The method of claim 1, wherein said administering comprises administering the liposome-entrapped compound at least about once every 28 days.

4. The method of claim 1, wherein said administering comprises administering the liposome-entrapped compound four times with at least about 28 days between administrations.

5. The method of claim 1, wherein said administering comprises administering liposomes having a surface coating of hydrophilic polymer chains.

6. The method of claim 1, wherein said administering comprises administering liposomes having a surface coating of polyethylene glycol polymer chains.

7. The method of claim 1, wherein said administering comprises intravenously administering.

8. The method of claim 1, wherein said administering comprises administering to a patient previously treated with a chemotherapeutic regimen comprised of cisplatin and paclitaxel.

9. The method of claim 8, wherein said administering comprises administering to a patient having a complete clinical response to said prior chemotherapeutic regimen.

10. The method of claim 1, wherein said administering comprises administering to a patient with advanced ovarian cancer.

11. The method of claim 10, wherein said administering comprises administering to a patient with Stage IIIC or Stage IV ovarian cancer.

12. A method for improving the progression-free interval or overall survival of a patient previously diagnosed with advanced ovarian cancer, suboptimally-debulked and previously treated with a platinum-based chemotherapeutic agent, comprising
administering an anthracycline compound entrapped in liposomes.

13. The method of claim 12, wherein said administering comprises administering doxorubicin entrapped in liposomes.

14. The method of claim 12, wherein said administering comprises administering liposomes having a surface coating of hydrophilic polymer chains

15. The method of claim 12, wherein said administering comprises administering the liposome-entrapped compound at least about once every 28 days.

16. The method of claim 12, wherein said administering comprises administering the liposome-entrapped compound four times with at least about 28 days between administrations.

17. The method of claim 12, wherein said administering comprises administering liposomes having a surface coating of polyethylene glycol polymer chains.

18. The method of claim 12, wherein said administering comprises intravenously administering.

19. The method of claim 12, wherein said administering comprises administering to a patient previously treated with a chemotherapeutic regimen comprised of cisplatin and paclitaxel.

20. The method of claim 19, wherein said administering comprises administering to a patient having a complete clinical response to said prior chemotherapeutic regimen.

21. The method of claim 12, wherein said administering comprises administering to a patient with advanced ovarian cancer.

22. The method of claim 21, wherein said administering comprises administering to a patient with Stage IIIC or Stage IV ovarian cancer.
